(19) United States
(12) Patent Application Publication
(10) Pub. No.: US 2025/0210161 A1
(43) Pub. Date: Jun. 26, 2025

(54) Systems and Methods for Digital Prescription Management

(71) Applicant: Rx Passport, Inc., Las Vegas, NV (US)

(72) Inventor: Elliot Friedman, Las Vegas, NV (US)

(73) Assignee: Rx Passport, Inc., Las Vegas, NV (US)

(21) Appl. No.: 18/989,851

(22) Filed: Dec. 20, 2024

Related U.S. Application Data
(60) Provisional application No. 63/613,995, filed on Dec. 22, 2023.

(51) Int. Cl. G16H 10/60; G16H 20/10

(52) U.S. Cl. CPC G16H 10/60; G16H 20/10

(57) ABSTRACT

Disclosed are various embodiments of a drug management system. In some examples, the drug management system may represent a centrally hosted system that integrates a mobile application and/or desktop software. The drug management system may maintain a centrally hosted prescription data store for a patient that includes a list of all the patient's current medication prescriptions that may have been prescribed by multiple providers across different provider networks. Some embodiments may aid patient compliance with prescriptions by identifying the bottle needed to be taken at a scheduled time using a reader of the system and alerts presented to a patient. Some embodiments may aid in reducing inappropriate drug interactions or redundancies by analyzing prescribed drugs and presenting alerts to pharmacists, physicians, or other providers, or to a patient or a patient's support network. Some embodiments enable presenting the list of the patient's current medication prescriptions to a provider.

### CROSS-REFERENCE TO RELATED APPLICATIONS

This application claims the benefit under 35 U.S.C. § 119(e) of U.S. Provisional Application No. 63/613,995, titled “Systems and Methods For Digital Prescription Management” and filed Dec. 22, 2023, the entire contents of which are incorporated herein by reference.

### BACKGROUND

Patients, particularly Seniors and the Chronically ill are commonly cared for by multiple physicians, other medical providers, and pharmacists for their varied medical conditions. Their providers, who may change from time to time, may prescribe medications for the patient independently from one another. As patients age and become sicker, these prescriptions may change and may increase in number.

### SUMMARY

In some aspects, the techniques described herein relate to a computer-implemented method including (1) maintaining, within a repository of medication information, a data store (e.g., a centralized data store) of prescriptions for medication currently prescribed to a patient by providers from different provider networks and (2) providing, to each of the providers, a provider portal that provides each provider with access to view the data store, add new prescriptions to the data store, and modify existing prescriptions within the data store. Additionally or alternatively, the computer-implemented method may include (1) receiving a request, from a provider treating the patient at a first provider network, for another provider treating the patient at a second provider network to update or cancel a prescription added to the data by the other provider and (2) communicating the request to the other provider at the second provider network. This could be done directly or through a third party such as a pharmacy.

In some examples, the computer-implemented method may include receiving, at a patient mobile device, a list of medications currently prescribed to the patient and presenting the list to a digital medical record of a provider (e.g., prior to or during a patient encounter) to be entered into the digital medical record as a record of current medications of the patient. In some examples, this digital method could be enhanced by having a bar code, RFID, or other reader on the phone that reads the bar code, RFID, or other technology that can identify medicines in the patient's prescription bottles. In certain embodiments, the computer-implemented method may include generating a medication schedule based on the data store of the patient's current prescriptions and using a smart phone to alert the patient of when to take their medicines (e.g., including a bar code, RFID, or other identification technology to identify which prescription bottle contains the right medicines).

Method and systems of the presently disclosed embodiments were isolated or otherwise manufactured in connection with the examples provided below. Other features and advantages will be apparent from the detailed description, and from the claims.

### BRIEF DESCRIPTION OF THE DRAWINGS

FIG. 1 is a block diagram of a prescription medication management system by which some embodiments may operate.

FIG. 2, FIG. 3, and FIG. 4 are flow charts of processes 200, 300, and 400, respectively, that may be implemented in some embodiments to manage prescription medications for optimizing patient outcomes.

FIG. 5 is an exemplary implementation of a computing device that may be used in a system implementing techniques described herein.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Patient nonadherence to taking prescribed medication is epidemic, with an estimated 50% of patients not taking their medications as prescribed. The cost of drug nonadherence in the United States is an estimated $100 to $300 billion annually in unnecessary hospitalizations, with deaths that likely exceed 125,000 patients annually. Seniors and chronically ill patients may be especially vulnerable due to having (1) a large number of prescribed medications (e.g., prescribed by multiple prescribing providers at different institutions), (2) a varied dosing for each prescribed medication (e.g., how many pills of each medication to take), (3) a varied timing for taking each prescribed medication (e.g., morning, evening, twice a day, once a day, etc.), and/or (4) frequent updates to prescriptions (e.g., due to a change in provider, a change in patient condition, etc.).

Any of these complexities may make it difficult or impossible for a patient to adhere to the patient's regimen of prescribed medications. In an attempt to help with the task of remembering one's prescriptions, some applications allow a patient to manually add each medication prescribed to the patient to a digital list. However, while such applications may give a patient a sense of security, remembering to update the list each time a new medication is prescribed, or an existing prescription is modified, may be challenging for the patient. As such, the list may quickly become inaccurate and/or incomplete, making the sense of security provided by such applications a false one.

Often, when a patient is treated by multiple different providers, the providers may be in different provider networks. Such a provider network may be a group of related providers that exchange administrative information and/or health information regarding patients, or may share administrative resources. In some examples, a provider network may correspond to an organization such as a practice or a hospital and/or a group of connected organizations that exchange information and/or share resources. Often, different provider networks may have different and independent medical records, including independent electronic medical records. Information regarding one patient's care may thus be scattered across multiple discrete electronic medical records. These systems often do not share information, for administrative or technical reasons and/or for reasons related to patient privacy regulations. As a result, when multiple providers are prescribing medications to a patient, there is often not any unifying view of the prescriptions for a patient. This is particularly disadvantageous given that prescriptions for a patient are often changing, with prescriptions being stopped/removed, added, or substituted (as discussed above).

As a result of these shortcomings of electronically maintained prescription records, it is often up to a patient to determine what medications the patient is taking. For example, when a patient visits a provider, the provider may ask the patient for a list of current prescriptions. The patient may have to remember each of their prescription. This manual task is error-prone, particularly for vulnerable populations (e.g., certain seniors and/or chronically ill patients) burdened with an ever-evolving prescription regimen from different providers.

When medications for a patient are prescribed by providers from different provider networks, a lack of access to prescription data between provider networks may make it difficult or impossible for providers to catch contraindications. Additionally, with no reliable list of network and non-network medications available to providers for each patient, it is easy to “double” up on the same medication, often causing disastrous effects for the patient. In cases in which a provider network maintains a digital list of prescriptions prescribed for a patient at that provider network, a provider may examine the list and feel a false confidence that prescribing a new medication for a patient is safe because there are no known drug interactions or duplications of drug functions between the new medication and the medications within the list, when in actuality the new medication is contraindicated because of a drug interaction or duplication of function with a medication prescribed for the patient by a provider at a different provider network.

Providers are aware that patient-provided lists of medications are inaccurate. This lack of information is a universally recognized problem in the healthcare system and has been for decades. In addition, it has severe consequences: There are an estimated 125,000 patient deaths from taking drugs that are contraindicated because of interactions with other drugs or comorbid conditions. These deaths from contraindication, poor adherence and dangerous “double” ups of similar functioning drugs (i.e., blood pressure lowering), could be resolved by information sharing, but the well-recognized problem has persisted for decades, even after widespread adoption of electronic medical records, with no clear solution.

In light of each of these barriers to prescription adherence by patients and safe prescribing by providers, the present disclosure describes various embodiments of a computer-implemented system that (1) maintains a digital data store of prescriptions for medication for a patient, across multiple providers and/or provider networks involved in the patient's care, (2) updates the data store when a change is made (e.g., a new prescription is added and/or an existing prescription is changed or removed), (3) enables the patient to access the data store (e.g., to view a complete and current list of all of the patient's prescriptions prescribed at any provider network involved in the patient's care), (4) enables a provider (e.g., physician, other medical providers, and pharmacists) involved in the patient's care to view the data store (e.g., to check a current list of the patient's current prescriptions, prescribed at different provider networks involved in the patient's care, e.g., for contraindications prior to prescribing a new medication), and/or (5) enables providers (including pharmacists) across different provider networks to digitally coordinate patient care (e.g., to avoid prescription of incompatible medications) either directly or through a third party such as a pharmacy and/or third party exchange.

Disclosed are embodiments of a drug management system. In some examples, the drug management system may represent a centrally hosted system that integrates a mobile application (e.g., installed on a patient's mobile device), and/or desktop software (e.g., installed on devices maintained at medical clinics and/or pharmacies). In some examples, the drug management system may maintain a centrally hosted prescription data store for a patient that includes a list of all the patient's current medication prescriptions (e.g., prescribed by multiple providers across different provider networks). The patient's prescription data store may include a variety of information relating to a current prescription that may be relevant to the patient and/or the patient's providers (e.g., a medication being prescribed, the date that the medication was originally prescribed and/or that a prescription for the medication was modified, a patient reaction to the medication, a dose and/or schedule for the medication, patient compliance by identifying the bottle needed to be taken at the scheduled time by a reader of the system, a disease targeted by the medication, and/or contact information for a provider who prescribed the medication).

The drug management system of some examples described herein may provide various individuals and/or institutions involved in the patient's care with access to the patient's prescription data store (e.g., providers, pharmacies, the patient, and/or a caregiver of the patient). Features that may be included in some examples described herein and that may be configured for each of these individuals and/or institutions are described below. As will be explained, these features may improve the functioning of a computer (e.g., by improving the accuracy of patient data maintained by a computer and/or improving accessibility of patient data to providers involved in the patient's prescription regimen). It should be appreciated that embodiments are not limited to including any or all of these features, and that some embodiments may include any one or more of these features.

As a first example, a drug management system may provide the patient's providers with access to the patient's prescription data store. By providing the patient's providers with access to the patient's prescription data store, the disclosed drug management system may provide the providers with the ability to review and analyze an accurate and/or current list of the patient's prescribed medications, which may include medications prescribed by providers at different provider network involved in the patient's care (e.g., when a patient receives care from multiple providers of different provider networks). A provider may, prior to prescribing the patient with a new medication, review the list to check whether the new medication may have a negative drug interaction with another medication the patient is currently taking, is duplicative in function (i.e. lowering blood pressure) with another medication, or otherwise use information on other medications the patient is taking to inform the provider's choice of whether to prescribe a medication, what medication to prescribe, and/or what dose or formulation of the medication to prescribe. Additionally, the drug management system may enable a provider to update the patient's prescription data store (e.g., by adding a new prescription to the patient's prescription data store and/or by modifying an existing prescription previously added by the provider to the patient's prescription data store by the provider). These updates may be reflected in the view of the patient's prescription data store provided to each of the patient's providers. In some examples, the drug management system may ensure that the data store remains up to date by updating the data store to include additions or modifications continually (e.g., at designated intervals) or occasionally (e.g., each time a change to the data store is detected).

In some embodiments in which the drug management system enables multiple providers to enter new prescriptions to the patient's prescription data store, a first provider entering a new prescription may notice an existing prescription in the patient's prescription data store, prescribed by a second provider, that has a known drug interaction with the new prescription or is duplicative in function. The drug management system may enable the first provider to notice the drug interaction in a variety of ways. In some examples, the drug management system may enable the first provider to notice the drug interaction or duplication by enabling the first provider to manually review the patient's current prescriptions (e.g., making the first provider aware of a prescription the first provider may otherwise not have known about). Additionally, the drug management system may automatically detect the drug interaction and/or duplication in function and present an alert—this can be done in various ways including reference to known interaction reference guides or through using an AI modules (e.g., using an AI-driven natural language processing tool) to profile the exact medications used by the patient and compare it with poor results from other similar patients taking the exact same regimen drugs; this could potentially be adjusted by age, sex, ethnicity or other demographic data. Importantly, the drug management system may enable the first provider to contact the second provider (e.g., by providing the first provider with contact information for the second provider and/or enabling the first provider to transmit a secure and HIPPA-compliant notification to the second provider requesting that the second provider remove and/or change the existing prescription) and/or may enable the first provider to contact an institution (e.g., a pharmacy, a non-profit, and/or a government agency) that coordinates safe prescribing across multiple providers or the initiating provider may put in the request directly to the other provider.

As a second example, the drug management system may provide a pharmacy with access to the patient's prescription data store, enabling the pharmacy to search the patient's list of current prescriptions for contraindications prior to dispensing a medication to the patient. As conventional approaches to presenting lists of medication limit a pharmacy to viewing only prescriptions filled at the pharmacy and/or a provider network that includes the pharmacy, this more comprehensive view of a patient's care and list of prescriptions can advantageously increase patient safety and reduce risk of negative drug interactions affecting a patient's health. In some embodiments, the drug management system may request and/or require a pharmacy to digitally acknowledge that the list of the patient's prescription medications has been reviewed by a pharmacist prior to dispensing a new prescription to the patient.

In one example, the drug management system may process (e.g., automatically) an order for a prescription, added by a provider to the patient's prescription data store, at a pharmacy (e.g., a pharmacy designated by the patient and/or the provider). By enabling the provider to digitally order a prescription as part of a process for digitally adding the prescription to the patient's data store of prescriptions, the drug management system may improve the functioning of a computer by improving computer efficiency. This efficiency may further improve the functioning of a computer by improving the accuracy of patient data maintained by the computer, as integrating the process for ordering a prescription with the process for adding the prescription to a centrally hosted data store may increase the likelihood that the data store will remain up to date.

In some examples, the drug management system may enable a pharmacy to digitally communicate with prescribing providers. For example, the drug management system may enable the pharmacy to send a message from a pharmacy portal of the drug management system to a provider portal of the drug management system with a concern or suggestion relating to a prescription. In some examples, the pharmacy portal may visually and/or logically associate, within an interface, a display of a prescription and a triggering element for sending a message to a provider who ordered the prescription. In one embodiment, the message may include a specific suggestion (e.g., changing to a different medication and/or adjusting a dosing) and a selectable element that, when selected at the provider portal, automatically updates the prescription as suggested by the pharmacy (e.g., within the patient's prescription data store). By facilitating digital communication between pharmacists and providers, the drug management system may digitally support a practice of safe prescribing.

As a third example, the drug management system may enable the patient to access the patient's prescription data store (e.g., with the patient's list of current prescriptions). In some examples, the patient's prescription data store may be presented to the patient via a mobile application installed on patient mobile device. Access to a digitally maintained list of current medications may be especially empowering to vulnerable populations with declining and/or compromised cognition (e.g., some senior populations) who manage many prescriptions that may change frequently (e.g., at each doctor's visit). This feature may relieve patients of the burden of remembering or manually tracking a list of current medications and may enable patients to easily provide a comprehensive list to each of the patient's new or existing providers (e.g., in place of listing off current medications from memory, bringing a bag of each current medication the patient is taking, or relying on an application that requires the patient to manually add each prescription, and/or modification to a prescription, to the application).

In some examples, as a start to the process of collecting a complete and reliable set of the patients medicines, the mobile or other digital device might have a label reader for the patient to point at each of their prescription bottles to load into the mobile device.

The drug management system may enable a patient to share the patient's current list of prescribed medications with a provider in a variety of ways. In examples in which the drug management system provides the list of prescribed medications to the patient via a mobile application, the patient may show the provider the list from the patient's mobile application. In one embodiment, the drug management system may include an automatic transfer feature (e.g., effected through a Bluetooth and/or other communication protocol) that enables an automatic transfer of the patient's current list of prescribed medications from the patient's mobile application to patient records software installed on a device at the provider's office. Additionally or alternatively, the drug management system may enable the patient to share the patient's current list of prescribed medications with a provider via a provider account registered with the drug management system. In one such example, the drug management system may enable the patient to digitally add a provider to the patient's account (e.g., via the mobile application), which may grant the provider with access to the patient's prescription data store via the provider account.

In examples in which the drug management system is integrated with an application installed on a patient mobile device or another digital device, the application may be configured with an alarm function that alerts the patient to take the medications currently prescribed in the patient's prescription data store (e.g., prompting the patient to take the correct medications at the correct times). The alert may include a snooze function for postponing the alert or an off function to shut off the alert after the patient has taken a medication. To further support the patient taking the correct medications, the alarm function may be keyed to specific bottles of medication. In such examples, a different identifier (e.g., a chip, barcode, and/or QR code) may have been placed on each bottle for a medication with an alert and the mobile application may, when alerting the patient to take a medication, prompt the patient to scan the identifier on the medication's bottle. If the correct identifier is scanned, the mobile application may provide a digital confirmation (e.g., via an alarm light turning green, an audio confirmation that the correct medication is being taken, and/or an alarm that only turns off in response to the correct identifier being scanned).

In some examples, the drug management system may facilitate the task of filling a prescription for a patient. For example, a provider adding a new prescription or a refill to the patient's prescription data store may trigger an electronic version of the prescription to be added to a patient mobile or other digital application of the drug management system. In this example, the patient may access the prescription via the patient mobile application to present to a pharmacy. Additionally or alternatively, the drug management system on the mobile phone or other digital device may enable a provider to send a prescription from a provider portal of the drug management system to a pharmacy portal of the drug management system (e.g., as part of adding the prescription to a patient's prescription data store).

In some examples, compliance with taking medicines might be tracked and/or communicated to a physician, other medical provider, and/or pharmacist. Patient compliance can be determined in a variety of ways. In one embodiment, once the scheduler on the mobile app or other digital device identifies the correct bottle to take, it may be assumed that the patient has actually taken the medicine and is “compliant.” The patient's compliance history with each medicine can be communicated directly with the physician, other medical provider, or pharmacist, and/or can be shown at the next scheduled appointment and/or interaction.

In some examples, access rights to prescription information maintained by the drug management system (e.g., medications a patient is prescribed and/or adherence information) may be configurable by the patient. For example, the drug management system may enable the patient to select to (1) share the patient's prescription data to any provider and/or pharmacy linked to the patient within the drug management system, (2) share the patient's prescription data selectively (e.g., selecting to only share prescription adherence information for a particular medication with a prescribing office associated with the particular medication), and/or (3) only maintain the patient's prescription information for personal consumption.

Described below are examples of ways in which techniques described herein may be implemented. It should be appreciated that these examples are merely illustrative, that embodiments are not limited to operating in accordance with the specific examples shown in the figures and discussed below, and that other embodiments are possible. The following examples are put forth so as to provide those of ordinary skill in the art with a complete disclosure and description of how to make and use the prescription management system and some aspects and embodiments herein and are not intended to limit the scope of the various aspects and embodiments herein.

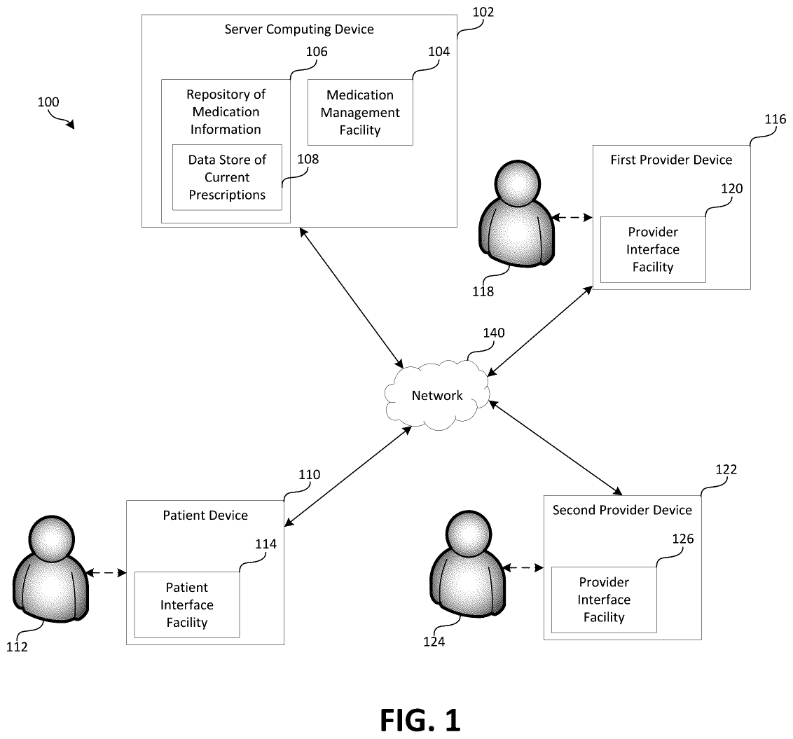

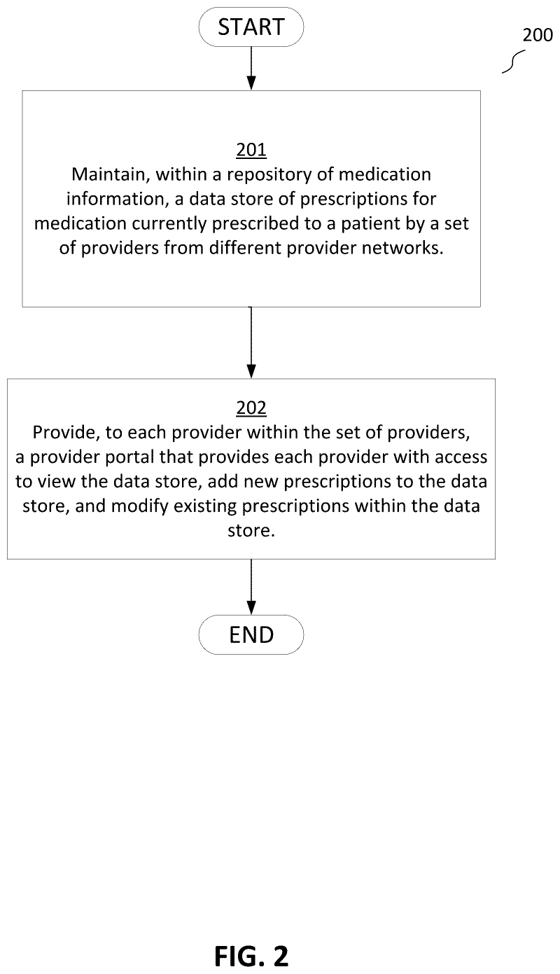

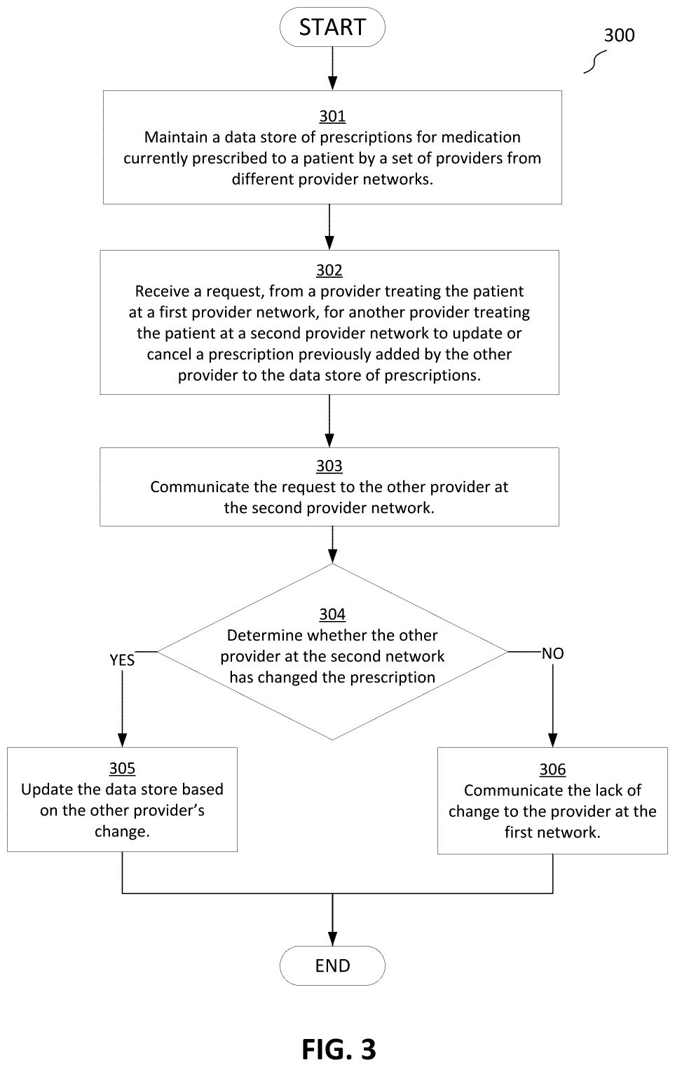

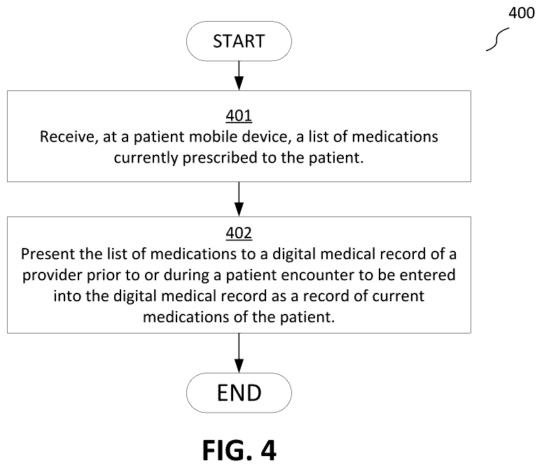

FIG. 1 illustrates a block diagram of a system 100 with which some embodiments may operate for managing patient prescriptions (e.g., involved in carrying out the methods of the disclosure that will be described in connection with FIGS. 3-5). As depicted in FIG. 1, system 100 can include a server computing device 102. Server computing device 102 may be any type or form of device (e.g., a backend computing device) that may perform functions directed at patient medication management. Server computing device 102 may include a repository of medication information 106. In some examples, the repository of information 106 may include a data store of current prescriptions 108 maintained for a particular patient (e.g., the patient 112). Additionally, system 100 can include a medication management facility 104 (e.g., configured to perform one or more of the steps described in connection with FIGS. 2-5). In some examples, the medication management facility 104 may be configured to maintain, update, and/or provide access to the data store of current prescriptions 108. Additionally or alternatively, the medication management facility 104 may be configured to analyze values or trends (e.g., derived from the data stored within data store of current prescriptions 108) for the patient 112. In some embodiments, the medication management facility 104 may receive information from, and/or output information to, the data store of current prescriptions 108 and/or a user device (e.g., a patient and/or provider device) via a user interface.

As also depicted in FIG. 1, the system 100 can include a patient device 110 (i.e., a user computing device), which may be a desktop or laptop personal computer, smart mobile phone, server, wearable device, or other suitable device used by the patient 112. The patient device 110 may include patient interface facility 114 (e.g., configured to perform one or more of the steps described in connection with FIGS. 2-5). In some examples, the patient interface facility 114 may generate, maintain, and/or present a user interface by which the patient 112 may interact with the patient device 110. In some examples, the user interface may represent a patient portal. In some examples, as will be described in greater detail in connection with FIGS. 2-5, the patient 112 can use the user interface to interface with the data store of current prescriptions 108 maintained for the patient 112 and/or medication management facility 104 of the server computing device 102. For example, the patient 112 may operate the user interface to access (e.g., view) the data store of current prescriptions 108, share the data store of current prescriptions 108 with a provider, and/or add a provider to a data store of providers with permission to access the data store of current prescriptions 108. The medication management facility 104 may output information (e.g., maintained within the data store of current prescriptions 108) to the patient 112 via the user interface. In some embodiments, the user interface may be provided via a mobile application installed on the patient device 110 and/or may include a web interface, such as one or more web pages which may display data maintained with the data store of current prescriptions 108, but embodiments are not so limited. The user interface may accept input in a variety of different formats, such as through speech recognition, text input, or other means, as embodiments are not limited in this respect.

In some examples, the system 100 can include one or more provider devices used by providers involved in the care of the patient 112 (e.g., a first provider device 116, of a first provider 118 with a provider interface facility 120, and a second provider device 122, of a second provider 124 with a provider interface facility 126). The provider devices may be any type of device used by a provider for patient care, such as a desktop, laptop, smart mobile phone, server, or other suitable device. The provider interface facility of each provider device may generate, maintain, and/or present a user interface by which a provider may interact with the provider device. In some examples, as will be described in greater detail in connection with FIGS. 2-5, a provider can user this user interface to interface with the data store of current prescriptions 108 (e.g., to view, add to, and/or modify prescriptions with the data store of current prescriptions 108) and/or to output communications to other providers involved in the care of the patient 112. In some examples, the user interface may represent a provider portal.

In some examples, the provider devices within system 100 may include devices of providers who practice within different provider networks. In these examples, the first provider device 116 of the first provider 118 may provide access to resources maintained by a first provider network (e.g., patient records including prescription records) that are not accessible to the second provider 124. Similarly, the second provider device 122 may provide access to resources maintained by a second provider network that are not accessible to the first provider 118. In contrast, the provider interface facilities 120 and 126 may enable both providers to access the data store of current prescriptions 108 maintained for the prescriptions of the patient 112.

As depicted in FIG. 1, the system 100 can include a network 140 to facilitate communications among the various devices within the system 100 (e.g., the server computing device 102, the patient device 110, one or more provider devices such as the first provider device 116 and the second provider device 122, and/). The network 140 can be or include any one or more wired and/or wireless, local- and/or wide-area network, including one or more enterprise networks and/or the Internet.

While the illustration of FIG. 1 includes the medication management facility 104 on server computing device 102, embodiments are not so limited. In other embodiments, the medication management facility 104 can be implemented on the patient device 110, the first provider device 116, the second provider device 122, and/or a combination of multiple of the devices depicted in FIG. 1. In some embodiments, the various user interfaces described herein may not be separate from the medication management facility 104, but instead may be implemented as a single program or software application.

FIG. 2, FIG. 3, and FIG. 4 provide flow charts of processes 200, 300, and 400, respectively, that may be implemented in some embodiments (e.g., by a system such as system 100 in FIG. 1) to manage a patient's prescriptions (e.g., prescribed to the patient by providers across multiple provider networks). Processes 200, 300, and 400 can be implemented in some embodiments by medication management facility 104, patient interface facility 114, provider interface facility 120, and/or provider interface facility 126, described in connection with FIG. 1, on a computer-readable storage media. Each of these processes will now be described, in turn.

### Data Store of Patient Prescriptions

Process 200 in FIG. 2 begins with step 201, where the system maintains, within a repository of medication information (e.g., the repository of medication information 106 in FIG. 1), a data store of prescriptions for medication (e.g., the data store of current prescriptions 108 in FIG. 1) currently prescribed to a patient (e.g., the patient 112 in FIG. 1) by a set of providers from different provider networks (e.g., the first provider 118 from a first provider network and the second provider 124 from a second provider network illustrated in FIG. 1).

The repository of medication information may be any type or form of medication information (e.g., for multiple users of a medication management service) stored in a data structure (e.g., a database). In some examples, the repository may include, for each patient for whom medication information is maintained, a data store of the patient's current prescriptions. The data store of the patient's current prescriptions may be any type or form of data structure that stores digital data. In some examples, the data store may represent an organized data structure that receives storage instructions/requests (e.g., from the facilities described in FIG. 1). The data store may correspond to any type of known or to be known storage. The data store may store a variety of data relating to the patient's prescriptions including, without limitation, a name of a medication being prescribed, dosing and/or timing information for the medication, a date on which the medication was originally prescribed and/or modified, an expiration date for the prescription, a condition targeted by the medication, a number of times a prescription for the medication has been filled, a number of refills left for the prescription, and/or information (e.g., contact information) relating to a pharmacy at which the medication has been filled, and/or a provider who prescribed the medication, or patient compliance measured by pointing the mobile or digital device at the correct scheduled prescription bottle. In some examples, the data store may only store data relating to current prescriptions (e.g., deleting and/or archiving data relating to prescriptions that have expired and/or been removed). In other examples, the data store may store data relating to any of the patient's prescriptions (current or past). In one embodiment, an organization of the data store may include multiple entries (one for each of the patient's prescriptions). Each entry may include the name of a mediation and data relating to the medication (e.g., the data described above such as the date on which the medication was originally prescribed and/or modified etc.).

### Provider Portal

The system may include a variety of features that ensure the accuracy of the patient's data store of current prescriptions (e.g., ensuring that the data store of current prescriptions is complete and up to date). In some examples, the system may ensure the accuracy of the patient's data store of current prescriptions by enabling each provider prescribing medications to the patient to access the data store. For example, as indicated at step 202, the system can provide, to each provider within the providers from the different provider networks prescribing medications for the patient, a provider portal that provides each provider with access to view the data store, add new prescriptions to the data store, and modify existing prescriptions within the data store.

The system may provide the provider portal to a provider in a variety of ways. In some examples, the system may instruct a provider device (e.g., first provider device 116 and/or second provider device 122 in FIG. 1) to present the provider portal as and/or within a user interface. In some examples, the system may maintain desktop software, installed on the provider device, configured to present the provider portal to the provider. The provider portal may be configured with any layout and/or flow for data retrieval and/or input that enables a provider to view the patient's data store of current prescriptions, add new prescriptions to the patient's data store of current prescriptions, and/or modify existing prescriptions with the patient's data store of current prescriptions. In one embodiment, the provider portal can be configured to output (e.g., for provider viewing) the prescriptions within the patient's data store of current prescriptions, receive new prescriptions to be added to the patient's data store of current prescriptions, and receive modifications to be applied to existing prescriptions in the patient's data store of current prescriptions. In some examples, the provider portal may also enable a provider to order a new prescription at a pharmacy. In one embodiment, the provider portal may include an order element that, when selected, initiates a prescription to be ordered at a designated pharmacy. In some embodiments, ordering the prescription via the provider portal may automatically trigger the prescription to be added to the patient's data store of current prescriptions.

In some examples, the system may analyze a new prescription, received from a provider to be added to the patient's data store of current prescriptions, against existing prescriptions (e.g., to identify any contraindications). If the system detects that the new prescription is contraindicated (e.g., because of a potential drug interaction with an existing prescription within the patient's data store of current prescriptions or already exists with the same function (e.g., blood pressure lowering)), the system may perform a digital circumvention. In some examples, the digital circumvention may include digitally alerting the prescribing provider, a pharmacist, the patient, or another party that the attempted new prescription is contraindicated (e.g., because of the potential drug interaction with an existing prescription or repeating the same functioning medicine) and/or presenting (e.g., within the digital alert) a suggestion for an alternate prescription that is not contraindicated. The system may determine contraindication using information indicating contraindicated medications, such as by querying a data store of information maintained locally by the system and/or by querying a service of information regarding medications or by developing, with an AI tool such as a natural language processing tool driven by AI, its own continuing learning database of the exact configuration of medicines for each patient adjusted for various demographics: age, sex, ethnicity, etc. In some such embodiments, the digital circumvention may include providing (e.g., within the patient's data store of current prescriptions) a digital note indicating the contraindication or double up on the same functioning prescription, the provider who authorized ignoring the contraindication, and/or a reason (received via user input from the provider) that the contraindication is being ignored.

In some examples, the rights of the patient's providers to modify existing prescriptions may be conditional. For example, the system may restrict each provider from modifying prescriptions for which the provider is not the prescribing provider (e.g., enabling each provider to modify only prescriptions for which the provider is the prescribing provider). In such examples, the system may enable a provider, who is restricted from modifying a prescription prescribed by another provider, to electronically coordinate modifying the prescription with the other provider (e.g., in instances in which a new prescription the provider is attempting to add to the data store is contraindicated by the existing prescription). Such a scenario will be described in more detail later in connection with FIG. 3. In other embodiments, the system may enable a pharmacist to modify the prescription. In such cases, upon receiving information via the system regarding a contraindicated prescription (e.g., because of an interaction or redundancy with another prescription), a pharmacist may consult with either prescribing provider (e.g., doctor) to attempt to resolve the contraindication, including modifying either prescription with input from the other providers.

### Patient Portal

In addition to providing the patient's providers with access to the patient's data store of current prescriptions (e.g., via the provider portal), the system may provide the patient with access to the patient's data store of current prescriptions via a patient portal (e.g., provided to the user via a mobile application installed on the patient device). The patient portal may present, to the patient, an identification (e.g., a list) of the prescriptions of the data store. In some examples, the system may restrict the patient from modifying the prescriptions (e.g., only enabling a provider who prescribed a prescription to modify the prescription). In other examples, the system may receive a request to modify a prescription from a patient and may update a prescription in response. In some embodiments, the system may enable the patient to scan a physical prescription and/or upload a digital prescription to the patient portal. In these embodiments, the system may identify a prescription scanned or uploaded to the patient device by the patient and may add the scanned and/or uploaded digital prescription to the patient's data store of current prescriptions in response to the scanning and/or uploading (e.g., including scanning existing patient prescription bottle labels).

In addition to presenting an identification of the patient's current prescriptions to the patient via a patient portal, in some examples the system may provide the patient with functionality aimed at helping the patient to adhere to the patient's prescription regime. For example, the system may include a mobile application installed on the patient device (e.g., the application through which the patient portal is accessed) with an alarm function. The alarm function may instruct the patient mobile device to present an alert (e.g., an alarm, a push notification, etc.) each time a medication within the patient's data store of current prescriptions is to be taken. The timing of the alerts may be based on timing information extracted from the patient's data store of current prescriptions. As a specific example, the system may extract timing information from the data store for (1) a prescription for a first medication prescribed by a provider of a first provider network (i.e., information indicating that the first medication is to be taken at a first moment in time) and (2) a prescription for a second medication prescribed by a provider of a second provider network (i.e., information indicating that the second medication is to be taken at a second moment in time). Then, in response to determining (based on the extracted information) that the patient is to take the first medication at the first moment in time and the second medication at the second moment in time, the system may instruct the patient mobile device to present (1) a first alert at the first moment in time (indicating that it is time to take the first medication) and (2) a second alert at the second moment in time (indicating that it is time to take the second medication). In some examples, an alert may include a snooze function for postponing an alert or an off function to shut off an alert (e.g., after the patient has taken a medication corresponding to the alert).

In some examples, the alarm function may be integrated with a medication verification function. In these examples, each time a medication within the data store of prescriptions is to be taken, in addition to instructing the patient mobile device to present an alert, the system may instruct the patient mobile device to digitally verify that a correct medication is being taken by (1) prompting the patient to present, to a camera of the patient mobile device, an identifier (e.g., a chip, barcode, and/or QR code) associated with (e.g., affixed to) a medication bottle and (2) scanning the identifier presented to the camera. The system may then determine whether the correct medication is being taken (e.g., based on whether the presented identifier matches the identifier keyed to the correct medication) and may instruct the patient mobile device to indicate the same (e.g., to digitally verify that the correct medication is being taken or to digitally warn that an incorrect medication is being taken). The patient mobile device may indicate whether the correct medication is being taken in a variety of ways (e.g., via a light turning green if the correct medication is being taken or red if the incorrect medication is being taken, via an alarm that only turns off in response to be correct identifier being scanned, via text presented via a user interface, etc.).

In some examples, the system may include patient functionality that eases the burden of filing a prescription (e.g., reducing the likelihood that a prescription will be lost). For example, a provider adding a new prescription or a refill to the patient's data store of current prescriptions may trigger an electronic version of the new prescription or refill to be added to the patient's patient portal. In these examples, the patient may access the electronic version via the patient portal (e.g., provided via the patient mobile application) to present to a pharmacy to have dispensed by the pharmacy.

In some examples, the system may enable the patient to configure access rights to the prescription information maintained within the data store of current prescriptions. As a specific example, the drug management system may enable the patient to select to (1) share the patient's prescription data to any provider and/or pharmacy linked to the patient within the drug management system (e.g., within a database of the patient's providers), (2) share the patient's prescription data selectively (e.g., selecting to only share prescription adherence information for a particular medication with a prescribing office associated with the particular medication), and/or (3) only maintain the patient's prescription information for personal consumption.

### Pharmacy Portal

In some examples, the system may provide access to the patient's data store of current prescriptions to a pharmacy. In some such examples, an order for a prescription (e.g., ordered via the provider portal described above) may be presented to the pharmacy via the pharmacy portal. In one embodiment, the pharmacy portal may enable the pharmacy to route digital messages to the provider portal of any provider at any provider network who is associated with the patient within the system (e.g., any provider who has been granted access to the patient's data store of current prescriptions). In one such embodiment, the pharmacy portal may visually and/or logically associate, within an interface, a display of a prescription and a triggering element for sending a message to a provider who ordered the prescription. In one embodiment, the message may include a specific suggestion (e.g., changing to a different medication and/or adjusting a dosing) and a selectable element that, when selected at the provider portal, automatically updates the prescription as suggested by the pharmacy (e.g., within the patient's prescription data store). In some examples, the pharmacy portal may require a member of the pharmacy team to digitally acknowledge that the patient's data store of current prescriptions has been reviewed (e.g., by a pharmacist) prior to dispensing a new prescription to the patient.

The pharmacy portal may have a variety of uses. For example, the pharmacy portal may be used as a “clearing house” for messages sent between doctors and medical providers (e.g., to request changes to a prescription based upon potential adverse events or double dosing, getting acceptance for the potential change, changing the patient's database of prescriptions, etc.).

### Digital Data Store Views and Data Store Maintenance

As discussed previously, the system may transmit an identification of the prescriptions of the patient's data store of current prescriptions to each of the user devices described above (e.g., the patient device, a provider device, and/or a pharmacy device via the patient, provider, and/or pharmacy portals described above). The identification of the prescriptions may include a variety of information. Examples of such information include, without limitation, any of the information stored in the patient's data store of current prescriptions (e.g., a name of a medication being prescribed, dosing and/or timing information for the medication, a date on which the medication was originally prescribed and/or modified, an expiration date for the prescription, a condition targeted by the medication, a number of times a prescription for the medication has been filled, a number of refills left for the prescription, and/or contact information for a pharmacy at which the medication has been filled and/or a provider who prescribed the medication). In some examples, the identification may identify only current prescriptions. In other examples, the identification may identify current prescriptions and expired and/or removed prescriptions. In some examples, one or more of the portals described above may provide various views of the prescription information stored within the patient's data store of current prescriptions (e.g., enabling users to toggle between the various views). In one such example, a first view may include an identification (e.g., a list) of only current prescriptions and a second view may include a list of all prescriptions (e.g., expired prescriptions, a modification history for prescriptions, etc.). In some such examples, the second view may include annotations indicating a status of a prescription (e.g., expired, current, modified, etc.).

The system may ensure that the patient's data store of prescriptions remains up to date in a variety of ways. In some examples, the system may update the data store when a change is made (e.g., each time a new prescription is added and/or an existing prescription is changed or removed). The system may update the data store continually (e.g., at designated intervals) and/or occasionally (e.g., each time a change to the data store is detected). The system may enable users to make changes to the patient's data store in a variety of ways (e.g., as will be explained presently in connection with FIG. 3).

### Provider to Provider Framework

Process 300 in FIG. 3 begins with step 301, where the system maintains a data store of prescriptions (e.g., data store of current prescriptions 108 in FIG. 1) currently prescribed to a patient (e.g., the patient 112 in FIG. 1) by a set of providers from different provider networks (e.g., the first provider 118 and the second provider 124 in FIG. 1). The data store of prescriptions may include any of the features described for the patient's data store of current prescriptions in connection with FIGS. 1 and 2.

At step 302, the system may receive a request, from a provider treating the patient at a first provider network (e.g., the first provider 118), for another provider treating the patient at a second provider network (e.g., the second provider 124) to update or cancel a prescription previously added by the other provider to the data store prescriptions. In some examples, prior to receiving the request, the system may have (1) received a request from the provider to enter a new prescription to the patient's data store of prescriptions, (2) analyzed the new prescription against existing prescriptions within the patient's data store of prescriptions for contraindications, (3) determined, as part of the analyzing, that the new prescription is contraindicated because of a known drug interaction with the existing prescription and/or double dosed by prescribing tow medications with similar functionality, and (4) presented (e.g., within an interface, such as the provider portal discussed in connection with FIG. 2, used by the provider to enter the new prescription) an alert indicating the contraindication. In these examples, the provider may have submitted the request in response to viewing the alert. In other examples, the provider may submit the request in response to viewing the patient's data store of prescriptions and manually identifying a concern. In some examples in which the analysis of new and existing prescriptions is automatic, the disclosed system may use an AI modules (e.g., an AI-driven such as CHAT GP to profile the exact medications used by the patient and compare it with poor results from other similar patients taking the exact same regimen drugs; this could potentially be adjusted by age, sex, ethnicity or other demographic data.

At step 303, the system may communicate the request to the other provider at the second provider network. The system may communicate the request in a variety of ways. In some examples, the system may route the request to the other provider's provider portal as a secure and HIPAA-compliant digital alert and/or message (e.g., provided within an inbox and/or a patient page of the provider portal associated with the patient). The request transmitted to the other provider may be a message originated from the system in response to the request of the provider that the other provider be contacted, or may be a message transmitted from the provider to the other provider. In another embodiment, the system may additionally or alternatively prompt a notification to be made to the other provider by an employee of the system. Such a notification may be an audio and/or video call (e.g., phone call or videoconference), text message, email, or other technique for notifying.

Any suitable information may be communicated to the other provided in step 303. The information that is communicated may include information identifying a patient, either directly identifying (e.g., name) or identifying in the system (e.g., a patient record number or other alphanumeric identifier). In some embodiments, the information that is communicated may include an identification of the prescription made by the other provider that is the subject of the request for change, where the identification of the prescription may include any of the prescription-related information described herein. The information may include the prescription that the provider wishes to newly prescribe to the patient that has triggered the request to the other provider, include any of the prescription-related information described herein. Medical information regarding the patient that has prompted the provider's interest in prescribing the new prescription may also be included, such as an identification of the medical condition or symptoms with which the patient has been diagnosed. The information that is communicated may, in some cases, include a message from the provider to the other provider, such as comments that the provider wishes to convey to the other provider.

At step 304, the system may determine whether the other provider at the second network has changed the prescription. The system may determine whether the other provider has changed the prescription in response to a variety of triggers (e.g., in response to a determined period of time having lapsed, in response to receiving a request for an update from the provider, and/or in response to the other provider changing the prescription). If the other provider has changed the prescription, the system may (at step 305) update the patient's data store of prescriptions based on the change (e.g., by removing the existing prescription from the data store, indicating within the data store that the existing prescription has been removed, modifying the prescription, and/or adding a substitute prescription to the data store). Additionally, in some examples, the system may, in response to determining that the other provider has changed the prescription, send an order to a pharmacy for the updated prescription. If the other provider has not changed the prescription, the system may (at step 306) communicate the lack of change to the provider at the first network.

In some examples, the system may receive a digital message from the other provider at the second network for the provider at the first network (e.g., indicating a reason that the second provider is reluctant to change the existing prescription and/or recommending an alternate prescription for the provider that is not contraindicated because of the existing prescription). In these examples, the system may communicate the digital message to the provider (e.g., by routing the digital message to the provider portal of the provider).

In addition to enabling the provider to digitally communicate with the other provider regarding the prescription prescribed by the other provider, in some examples, the system may enable the provider to digitally communicate with an institution (e.g., a pharmacy, a third party service provider, a non-profit, and/or a government agency) that coordinates safe prescribing across multiple providers. In one such embodiment, the provider portal may include (e.g., proximate to an alert indicating a contraindication because of the existing prescription and/or proximate to an identification of the existing prescription) a selectable element for sending a request to the institution for help coordinating with the other provider to have the other provider remove or change the existing prescription.

### Patient to Provider Framework

In some examples, the disclosed system may enable the patient to digitally share the patient's data store of current prescriptions with a provider. At step 401 of process 400 in FIG. 4, the patient mobile device may receive (e.g., via a mobile application from a server such as server computing device 102 in FIG. 1), a list of medications currently prescribed to the patient (e.g., the medications listed with the patient's data store of current prescriptions). At step 402, the system may present the list of medications to a provider device (e.g., to a digital medical record of a provider accessed via the provider device) prior to or during a patient encounter (e.g., to be entered into the digital medical record as a record of current medications of the patient). The digital medical record may be a record maintained by the drug management system that maintains the patient's data store of current prescriptions and/or a record maintained by a third-party electronic record system.

In some examples, the system may present the list to the digital medical record in response to receiving a request from the patient mobile or other digital device (e.g., via the patient portal) to transmit the list to the provider device. In one such example, the system may present, within an interface of the patient mobile device (e.g., an interface associated with the patient portal), a share-initiation element that initiates sharing the patient's data store of prescriptions with a designated provider when selected via user input. In this example, the system may transmit the list of the prescriptions to the provider device in response to receiving user input selecting the share-initiation element. In one embodiment, the share-initiation element may represent a wireless-transfer element. In this embodiment, the system may wirelessly transmit the identification of the prescriptions to the provider device via a wireless radio frequency protocol. Such a wireless protocol may include a near field communication (NFC) protocol such as Radio Frequency Identification (RFID), a wireless personal area network (WPAN) protocol such as one of various versions of Bluetooth (e.g., Bluetooth Low Energy (BLE)), a wireless local area network (WLAN) protocol such as one of various versions of IEEE 802.11 (e.g., 802.11n), a wireless wide area network (WWAN) or wireless metropolitan area network (WMAN) network such as a cellular network, or other network. Embodiments are not limited to operating with any particular wireless communication protocol.

In some examples, the system may share the patient's data store of current prescriptions with a provider based on a provider designation received from the patient. For example, the system may (1) receive, from the patient (e.g., via the patient portal) a request to digitally add a provider to a data store of providers with permission to access the patient's data store of current prescriptions and (2) provide the provider with access to the patient's data store of current prescriptions in response to receiving the request. In some such examples, the system may receive requests to add multiple providers (each from a different provider network) and may provide each of the providers with access to the patient's data store of current prescriptions in response to receiving the requests.

### Adherence Monitoring

In some embodiments, the system may monitor the patient's adherence to the patient's prescription regime (as defined by the patient's data store of prescriptions). In one such embodiment, the system may detect a metric of patient adherence to a prescription within the patient's data store of prescriptions and add this metric to the repository of medication information and/or the patient's data store of prescriptions (e.g., within an adherence column and/or adhere element of an entry corresponding to the prescription and/or within a separate adherence report). The adherence data may be provided to any of the users described here (e.g., a provider via the provider portal, the patient via the patient portal, and/or the pharmacy via the pharmacy portal). The adherence data may be provided in real-time and/or on a periodic basis. In some examples, the patient may be provided with an interface for selecting which users are given access to adherence data. The system may monitor patient adherence in a variety of ways. In some examples, the system may deduce patient adherence based on refill information (e.g., collected from a pharmacy filling a prescription). In other examples, the system may deduce patient adherence based on data received from the mobile phone or digital app (e.g., that measures the systems function of patients digitally identifying prescription bottles to be taken at a certain schedule). ILLUSTRATIVE

### Computer Systems

Techniques operating according to the principles described herein may be implemented in any suitable manner. Included in the discussion above are a series of flow charts showing the steps and acts of various processes for patient prescription management. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionalities may be implemented in a single functional facility. It should also be appreciated that, in some implementations, some of the functional facilities described herein may be implemented together with or separately from others (i.e., as a single unit or separate units), or some of these functional facilities may not be implemented.

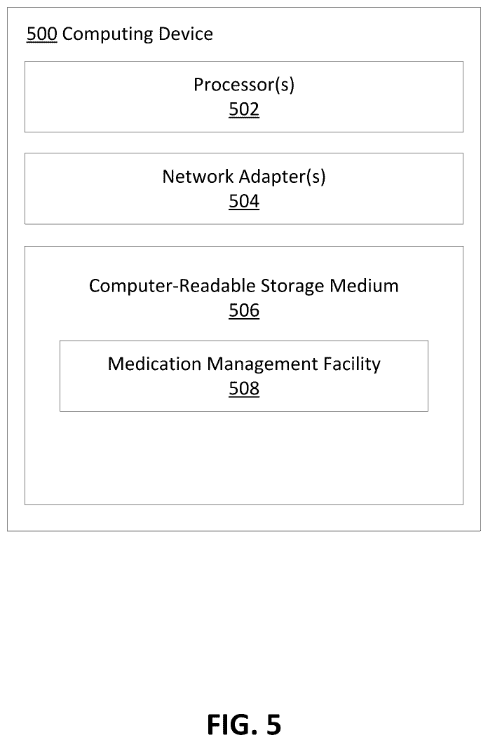

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 506 of FIG. 5 described below (i.e., as a portion of a computing device 500) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 1, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing devices (co-located or geographically distributed) dedicated to executing the techniques described herein, one or more Field-Programmable Gate Arrays (FPGAs) for carrying out the techniques described herein, or any other suitable system.

FIG. 5 illustrates one exemplary implementation of a computing device in the form of a computing device 500 that may be used in a system implementing techniques described herein, although others are possible. It should be appreciated that FIG. 5 is intended neither to be a depiction of necessary components for a computing device to execute one or more of the facilities described in connection with FIG. 1 in accordance with the principles described herein, nor a comprehensive depiction.

Computing device 500 may comprise at least one processor 502, a network adapter 504, and a computer-readable storage medium 506. Computing device 500 may be, for example, a desktop or laptop personal computer, a personal digital assistant (PDA), a smart mobile phone, a server, a wireless access point or other networking element, or any other suitable computing device. Network adapter 504 may be any suitable hardware and/or software to enable the computing device 500 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 506 may be adapted to store data to be processed and/or instructions to be executed by processor 502. Processor 502 enables processing of data and execution of instructions. The data and instructions may be stored on the computer-readable storage medium 506.

The data and instructions stored on the computer-readable storage media 506 may comprise computer-executable instructions implementing techniques which operate according to the principles described herein. In the example of FIG. 5, the computer-readable storage medium 506 stores computer-executable instructions implementing various facilities and storing various information as described above. The computer-readable storage medium 506 may store one or more of the facilities depicted in FIG. 1, which may implement one or more of the techniques described herein, such as medication management facility 508.

While not illustrated in FIG. 5, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

### Other Embodiments

From the foregoing description, it will be apparent that variations and modifications may be made to the embodiments described herein to adapt it to various usages and conditions. Such embodiments are also within the scope of the following claims.

It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting. Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

While some embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments described herein, or combinations of one or more of these embodiments or aspects described therein may be employed in practicing the present disclosure. It is intended that the following claims define the scope of the present disclosure and that methods and structures within the scope of these claims and their equivalents be covered thereby.

What is claimed is:

1. A computer-implemented method comprising:
providing, to a patient mobile device of a patient from a repository of medication information for a plurality of patients, a list of medications currently prescribed to the patient; and
presenting the list of medications to a digital medical record of a provider prior to or during a patient encounter to be entered into the digital medical record as a record of current medications of the patient.

2. The computer-implemented method of claim 1, wherein the list of medications comprises medications prescribed to the patient by a plurality of providers from different provider networks.

3. The computer-implemented method of claim 1, wherein presenting the list of medications to the digital medical record comprises wirelessly transferring the list of medications from the patient mobile device to the digital medical record.

4. The computer-implemented method of claim 3, wherein wirelessly transferring the list of medications from the patient mobile device to the digital medical record comprises:
presenting, within an interface of the patient mobile device, a wireless-transfer initiation element;
receiving user input selecting the wireless-transfer initiation element; and
wirelessly transferring the list of medications from the patient mobile device to a computing device of the provider for entry into the digital medical record, in response to receiving the user input.

5. The computer-implemented method of claim 1, wherein the digital medical record is maintained by at least one of:
a drug management system that maintains the repository; or
a third-party electronic medical record system.

6. The computer-implemented method of claim 1, wherein the list of medications is stored in a centrally hosted data store.

7. A computer-implemented method comprising:
maintaining, within a repository of medication information for a plurality of patients, a data store of prescriptions for medication currently prescribed to a patient by a plurality of providers from different provider networks; and
receiving, at the repository, a request, from a provider treating the patient at a first provider network, for another provider treating the patient at a second provider network to update or cancel a prescription previously added to the repository of medication for the patient.

8. A computer-implemented method comprising:
maintaining, within a repository of medication information, a data store of prescriptions for medication currently prescribed to a patient by a plurality of providers from different provider networks; and
providing, to each provider within the plurality of providers from different provider networks, a provider portal that provides each provider with access to view the data store, add new prescriptions to the data store, and modify existing prescriptions within the data store.

9. The computer-implemented method of claim 8, further comprising transmitting to a patient mobile device an identification of the prescriptions of the data store, prescribed to the patient by the plurality of providers from different provider networks.

10. The computer-implemented method of claim 9, further comprising transmitting the identification of the prescriptions of the data store to a computing device of the provider.

11. The computer-implemented method of claim 10, wherein transmitting the identification of the prescriptions to the computing device of the provider comprises transmitting the identification of the prescriptions to the computing device of the provider in response to a request received from the patient mobile device to transmit the identification to the computing device of the provider.

12. The computer-implemented method of claim 10, wherein transmitting the identification of the prescriptions to the computing device of the provider comprises transmitting the identification of the prescriptions to the computing device of the provider from the patient mobile device.

13. The computer-implemented method of claim 8, further comprising:
receiving, from the patient via a patient portal, a request to digitally add, to a data store of providers with permission to access the patient's data store of prescriptions, a first provider, from a first provider network, and a second provider, from a second provider network; and
providing the first and second providers with access to the patient's data store of prescriptions, via the provider portal, in response to receiving the request from the patient via the patient portal.

14. The computer-implemented method of claim 8, further comprising restricting each provider within the plurality of providers from modifying prescriptions for which the provider is not the prescribing provider.

15. The computer-implemented method of claim 14, further comprising:
detecting that a new prescription, which a first provider of a first provider network is attempting to add to the patient's data store of prescriptions, is contraindicated because of an existing prescription that was added to the patient's data store of prescriptions by a second provider of a second provider network; and
in response to detecting that the new prescription is contraindicated because of the existing prescription, performing a digital circumvention.

16. The computer-implemented method of claim 15, wherein the digital circumvention comprises digitally alerting the first provider of the first provider network that the attempted new prescription is contraindicated because of the existing prescription added to the data store by the second provider of the second provider network.

17. The computer-implemented method of claim 15, wherein the digital circumvention comprises restricting the first provider of the first provider network from adding the new prescription to the patient's data store of prescriptions.

18. The computer-implemented method of claim 15, wherein the digital circumvention comprises providing a digital prompt to the first provider of the first provider network suggesting an alternate prescription that is not contraindicated.

19. The computer-implemented method of claim 15, wherein the digital circumvention comprises enabling the first provider of the first provider network to contact the second provider of the second provider network to request that the second provider of the second provider network modify or remove the existing prescription.

20. The computer-implemented method of claim 19, further comprising:
receiving, from the first provider of the first provider network, a digital message for the second provider of the second provider network, the digital message comprising a request to modify the existing prescription; and
in response to receiving the digital message, routing the digital message to the second provider of the second provider network.

21. The computer-implemented method of claim 8, further comprising:
detecting a metric of patient adherence to a prescription within the patient's data store of prescriptions; and
adding the metric of patient adherence to the repository of medication information.

22. The computer-implemented method of claim 21, wherein detecting the metric of patient adherence comprises detecting the metric of patient adherence based on refill information collected from a pharmacy filling the prescription.

23. The computer-implemented method of claim 8, wherein:
the method further comprises providing, to a pharmacy, a pharmacy portal that provides access to the patient's data store of prescriptions prescribed to the patient by the plurality of providers from different provider networks; and
the provider portal presents, to a provider, an order element that, when selected, initiates a prescription to be ordered at the pharmacy.

24. The computer-implemented method of claim 23, wherein the pharmacy portal enables the pharmacy to route digital messages to any provider within the plurality of providers from different provider networks to be received via the provider portal.

25. The computer-implemented method of claim 8, further comprising instructing a patient mobile device to present an alert each time a medication within the patient's data store of prescriptions is to be taken, wherein instructing the patient mobile device to present the alert each time a medication is to be taken comprises:
determining, based on timing information maintained in the data store for a first prescription for a first medication prescribed by a first provider of a first provider network, that the patient is to take the first medication at a first moment in time;
determining, based on timing information maintained in the data store for a second prescription for a second medication prescribed by a second provider of a second provider network, that the patient is to take the second medication at a second moment in time; and
in response to determining that the patient is to take the first medication at the first moment in time and the second medication at the second moment in time, instructing the patient mobile device to present, at the first moment in time, a first alert that it is time to take the first medication and to present, at the second moment in time, a second alert that it is time to take the second medication.

26. The computer-implemented method of claim 25, further comprising, each time a medication within the data store of prescriptions is to be taken, in addition to instructing the patient mobile device to present an alert, instructing the patient mobile device to digitally verify that a correct medication is being taken, wherein digitally verifying that the correct medication is being taken comprises:
prompting the patient to present an identifier, affixed to a medication bottle, to a camera of the patient mobile device; and
scanning the identifier presented to the camera.

27. The computer-implemented method of claim 26, wherein digitally verifying that a correct medication is being taken further comprises at least one of:
digitally indicating that the correct medication is being taken in response to determining that the identifier corresponds to the medication; or
digitally indicating that an incorrect medication is being taken in response to determining that the identifier corresponds to another medication.

28. The computer-implemented method of claim 8, wherein the data store comprises, for a prescription for a medication within the data store, at least one of:
a name of the medication;
dosing information for the medication;
a patient reaction to the medication;
timing information for taking the medication;
a date on which the medication was originally prescribed;
a date on which the prescription was modified;
an expiration date for the prescription;
a condition targeted by the medication;
a number of times the prescription has been filled;
a number of refills left for the prescription;
contact information for a pharmacy at which the medication has been filled; or
contact information for a provider who prescribed the medication.

29. At least one computer-readable storage medium having encoded thereon executable instructions that, when executed by at least one processor, cause the at least one processor to carry out a method comprising:
maintaining, within a repository of medication information, a data store of prescriptions for medication currently prescribed to a patient by a plurality of providers from different provider networks; and
providing, to each provider within the plurality of providers from different provider networks, a provider portal that provides each provider with access to view the data store, add new prescriptions to the data store, and modify existing prescriptions within the data store.

30. An apparatus comprising:
at least one processor; and
at least one computer-readable storage medium having encoded thereon executable instructions that, when executed by the at least one processor, cause the at least one processor to carry out a method comprising:
maintaining, within a repository of medication information, a data store of prescriptions for medication currently prescribed to a patient by a plurality of providers from different provider networks; and
providing, to each provider within the plurality of providers from different provider networks, a provider portal that provides each provider with access to view the data store, add new prescriptions to the data store, and modify existing prescriptions within the data store.
